(19) United States
(12) Patent Application Publication
(10) Pub. No.: US 2024/0335521 A1
(43) Pub. Date: Oct. 10, 2024

(54) STABILIZATION OF ADJUVANTED VACCINE COMPOSITIONS AND THEIR USE

(71) Applicant: Vaxcyte, Inc., San Carlos, CA (US)

(72) Inventors: Christopher Iain GRAINGER, San Diego, CA (US); Sandrine BARBANEL, San Diego, CA (US); Shashwati Ujwalkumar ATWE, Fremont, CA (US)

(21) Appl. No.: 18/591,479

(22) Filed: Feb. 29, 2024

Related U.S. Application Data
(63) Continuation of application No. PCT/US2022/075859, filed on Sep. 1, 2022.
(60) Provisional application No. 63/240,244, filed on Sep. 2, 2021.

(51) Int. Cl. A61K 39/09; A61K 39/00; A61K 39/095; A61K 39/102; A61K 39/39; A61K 47/02

(52) U.S. Cl. CPC A61K 39/092; A61K 39/095; A61K 39/102; A61K 39/39; A61K 47/02; A61K 2039/55505; A61K 2039/6031; A61K 2039/6087

(57) ABSTRACT

The present disclosure provides stabilized vaccine compositions that resist the formation of unsuitable adjuvant flocculant or aggregates. The present disclosure further provides methods of using such compositions to induce immune responses against infections in subjects.

### CROSS-REFERENCE TO RELATED APPLICATIONS

This application claims the benefit of U.S. Provisional Application No. 63/240,244, filed Sep. 2, 2021, the disclosure of which is hereby incorporated by reference in its entirety.

### TECHNICAL FIELD

This invention relates generally to stabilized vaccine compositions and their use in preventing disease.

### DESCRIPTION OF THE TEXT FILE SUBMITTED ELECTRONICALLY

The sequence listing associated with this application is provided in text format in lieu of a paper copy, and is hereby incorporated by reference into the specification. The name of the text file containing the sequence listing is STRO_018_01WO_SeqList_ST26.txt. The text file is 6.17 kb, was created on Aug. 30, 2022, and is being submitted electronically via patent Center.

### BACKGROUND

Adjuvants are often added to vaccine formulations to enhance the immune response to the administered antigens. Aluminum-based adjuvants have been widely used in the pharmaceutical industry due to their effectiveness, low manufacturing cost, and proven safety profiles. Typical FDA-approved aluminum adjuvants (collectively, “alum”) include aluminum phosphate (AlPO₄) and aluminum hydroxide [Al(OH)₃].

Alum suspensions typically experience sedimentation, whereby the alum or complexes thereof settle at the bottom of a vaccine vial or syringe, or experience aggregation and/or flocculation of the alum. A manufactured drug product (DP) must be re-homogenized and exhibit adequate re-suspension of the alum prior to administration. An alum-based DP that cannot re-suspend from the bottom of a vial or pre-filled syringe, or one that either remains as aggregates of alum or presents itself as a flocculated suspension, exhibits non-ideal and unsatisfactory suspension characteristics for manufacturing and clinical use.

Re-suspension characteristics of alum-containing vaccines are typically driven through charge-based interactions, with some other forces potentially playing a role (e.g., hydrophobic, Van der Waals). The overall charge of a vaccine system is primarily driven by the pH of the solution that the alum is formulated in. A system where the solution pH renders the alum and/or alum-antigen complex in a neutral state reduces the repulsive nature of the system such that significant alum-antigen interactions take place, leading to flocculated systems (e.g., loose aggregates of alum/antigens in solution) unsuitable for administration. A solution pH that is substantially (e.g., >1 pH unit) different from the pH that renders the alum and/or the alum-antigen complex in a charged state creates a repulsive environment, wherein the alum-antigen complexes may repel one another, potentially leading to acceptable resuspension characteristics. However, dramatically altering the pH can also lead to degradation of certain antigens, including those discussed herein. Altering pH is therefore not always an adequate solution to achieving suitable resuspension characteristics.

Modulating the salt (e.g., NaCl) content of a vaccine DP can affect the charge profile of the alum or alum-antigen complexes and can therefore impact flocculation and aggregation. For instance, increasing salt concentration may reduce aggregate formation. However, high sodium chloride concentrations can lead to undesirable side effects upon administering the vaccine to a subject (e.g., pain at the injection site).

Accordingly, new methods are needed to prevent unwanted aggregation of aluminum adjuvant complexes in vaccine formulations without deviating from acceptable pH and osmolality.

### SUMMARY

Generally, a stabilized vaccine composition may include at least one polypeptide-polysaccharide conjugate, wherein the polypeptide component of the at least one polypeptide-polysaccharide conjugate comprises at least two non-natural amino acids (nnAA). The stabilized vaccine composition may include an aluminum adjuvant, a non-aluminum phosphate salt, and sodium chloride. The concentration of sodium chloride may be less than 200 mM, and the pH may be between about 5.5 and 7.

Also provided herein are methods of inducing a protective immune response against an infectious pathogen in a subject, comprising administering a stabilized vaccine composition to the subject. Methods may also include immunizing a subject against pneumococcal infection, comprising administering a stabilized vaccine composition.

Stabilized vaccine compositions may also be used in the manufacture of a medicament for inducing a protective immune response against a pathogen or against pneumococcal infection in a subject.

Generally, a method of stabilizing a vaccine composition may comprise mixing a non-aluminum phosphate salt with a solution comprising at least one polypeptide-polysaccharide conjugate, wherein the polypeptide component of the at least one polypeptide-polysaccharide conjugate comprises at least two non-natural amino acids (nnAAs). The solution may also comprise an aluminum adjuvant and sodium chloride. The concentration of sodium chloride may be less than 200 mM, and the pH may be between about 5.5 and 7

### BRIEF DESCRIPTION OF THE DRAWINGS

FIG. 1A shows a flocculated vaccine composition unsuitable for administration and FIG. 1B shows a resuspended stabilized vaccine composition.

FIG. 2 shows the visual effect of phosphate on settling time of non-stabilized and stabilized compositions.

FIG. 3 shows the sedimentation velocity of drug product with (stabilized) and without (non-stabilized) sodium phosphate.

FIG. 4 shows the particle size distribution of drug product with and without sodium phosphate.

FIG. 5 shows the composition of several test formulations of stabilized polypeptide-polysaccharide conjugate vaccines.

FIG. 6 shows the sedimentation velocity of several test formulations of stabilized polypeptide-polysaccharide conjugate vaccines.

### DETAILED DESCRIPTION

Described herein are stabilized vaccine compositions, methods of stabilizing vaccine compositions, and methods of inducing a protective immune response using stabilized vaccine compositions. “Stabilized” vaccine compositions may refer to ones that comprise an adjuvant (e.g., aluminum adjuvant) and are resistant to the formation of aggregates or flocculants (e.g., adjuvant, adjuvant-antigen) at a pH and osmolality suitable for manufacture, stable storage and administration to a subject.

### Polypeptide-Polysaccharide Conjugates

Generally, stabilized vaccine compositions described herein may comprise at least one polypeptide-polysaccharide conjugate. The polypeptide component of the at least one polypeptide-polysaccharide conjugate may contain non-natural amino acids (nnAA) in order to allow the polypeptide component to be coupled to the polysaccharide component. The polypeptide-component may, for instance, comprise at least two nnAA. This means that at least two separate or concurrent conjugation reactions may be effected per polypeptide component. Exemplary polypeptides are shown in Table 7.

The polypeptide component of the at least one polypeptide-polysaccharide conjugate may be a polypeptide antigen. In some embodiments, the at least one polypeptide-polysaccharide conjugate may be a substituted polypeptide antigen. As used herein, “polypeptide antigen” refers to a polypeptide comprising one or more epitopes that elicit an immunological response. Polypeptide antigens may be naturally occurring polypeptides, a mutant of a naturally occurring polypeptide (i.e., a protein that has one or more additions, deletions, or substitutions), or a truncated version of a naturally occurring polypeptide. As used herein, a “substituted polypeptide antigen” is a polypeptide antigen that comprises two or more nnAAs. nnAAs include a “click chemistry reactive group” (e.g. an azido group, an alkene group, an alkyne group, a 1,2,4,5-tetrazine group) which is suitable for a metal-free “click” chemistry reaction with a functional group on the polysaccharide of the polypeptide-polysaccharide conjugate. As used herein, an “unsubstituted polypeptide antigen” is a polypeptide antigen that does not comprise nnAAs containing functional groups suitable for a metal-free “click” chemistry reaction, and is not conjugated to a polysaccharide or additional polypeptide antigen.

In some embodiments, the polypeptide component of the at least one polypeptide-polysaccharide conjugate may be a carrier protein. As used herein, “carrier protein” refers to a non-toxic or detoxified polypeptide containing a T-cell activating epitope that is able to be attached to an antigen (e.g., a polysaccharide) to enhance the humoral response to the conjugated antigen in a subject and which contains at least two nnAAs. The term includes any of the bacterial proteins used as epitope carriers in FDA-approved vaccines. For instance, a carrier protein may be, or may be derived from, native Corynebacterium diphtheriae toxin, Clostridium tetani tetanospasmin, Haemophilus influenzae protein D (PD, HiD), an outer membrane protein complex of serogroup B meningococcus (OMPC), CRM197, or malaria ookinete specific surface protein Pfs25 substituted with two or more nnAAs. nnAAs include a “click chemistry reactive group” (e.g. an azido group, an alkene group, an alkyne group, a 1,2,4,5-tetrazine group) which is suitable for a metal-free “click” chemistry reaction with a functional group on the polysaccharide of the polypeptide-polysaccharide conjugate.

Herein, a “click chemistry reactive group” refers to a moiety, such as an azide or an alkyne, capable of undergoing a metal-free click chemistry reaction with a second click chemistry reactive group. In some embodiments, one click chemistry reactive group reacts with a second click chemistry reactive group to form a substituted triazole. Examples of this type of click reaction can be found, for instance, in International PCT Publication No. WO 2018/126229, PCT Application No. PCT/US21/018402, and US Publication No. 2020/0054739, incorporated herein by reference in their entirety and specifically with respect to disclosure of click chemistry reactions. General examples of metal-free click reactions used in biomedical applications can be found, for instance, in Kim, et al., Chemical Science, 2019, 10, 7835-7851, also incorporated hereby by reference in its entirety. Examples of nnAAs comprising click chemistry reactive groups include 2-amino-3-(4-azidophenyl)propanoic acid (pAF), 2-amino-4-azidobutanoic acid, 2-azido-3-phenylpropionic acid, 2-amino-3-azidopropanoic acid, 2-amino-3-(4-(azidomethyl)phenyl)propanoic acid (pAMF), 2-amino-3-(5-(azidomethyl)pyridin-2-yl)propanoic acid, 2-amino-3-(4-(azidomethyl)pyridin-2-yl)propanoic acid, 2-amino-3-(6-(azidomethyl)pyridin-3-yl)propanoic acid, and 2-amino-5-azidopentanoic acid. In some embodiments, the polypeptide component comprises two or more nnAAs, wherein at least one of the nnAAs is pAMF. In some embodiments, the polypeptide component comprises at least two nnAAs, wherein each of the at least two nnAAs are pAMF. In some embodiments, each of the nnAAs present is pAMF. It should be noted that nnAA may be incorporated into polypeptides described herein either by insertion (e.g., increasing the total number of amino acids over the original sequence), or by replacement of an existing amino acid (e.g., one or more lysine, arginine, or phenylalanine residues). In some embodiments, the nnAAs are substituted for lysine residues

In some embodiments, the at least two nnAAs are 2-amino-3-(4-azidophenyl)propanoic acid (pAF), 2-amino-4-azidobutanoic acid, 2-azido-3-phenylpropionic acid, 2-amino-3-azidopropanoic acid, 2-amino-3-(4-(azidomethyl)phenyl)propanoic acid (pAMF), 2-amino-3-(5-(azidomethyl)pyridin-2-yl)propanoic acid, 2-amino-3-(4-(azidomethyl)pyridin-2-yl)propanoic acid, 2-amino-3-(6-(azidomethyl)pyridin-3-yl)propanoic acid, or 2-amino-5-azidopentanoic acid.

It should be understood that the stabilized vaccine compositions described herein may comprise polypeptide-polysaccharide conjugates with varying numbers of nnAA, and therefore varying numbers of polysaccharide components conjugated to a single polypeptide component. The polypeptide component of the at least one polypeptide-polysaccharide conjugate comprises at least two non-natural amino acids (nnAA). In some embodiments, the polypeptide component comprises 3-8 nnAA. In some embodiments, the polypeptide component of the polypeptide-polysaccharide conjugate comprises 2, 3, 4, 5, 6, 7, or 8 nnAA. In some embodiments, the polypeptide component of the polypeptide-polysaccharide conjugate comprises 2, 3, 4, 5, 6, 7, or 8 nnAA, and the nnAA present are all the same. In some embodiments, the polypeptide component of the polypeptide-polysaccharide conjugate comprises 2, 3, 4, 5, 6, 7, or 8 nnAA, and the nnAA are all pAMF. In some embodiments, one or more nnAA incorporated into the polypeptide component of a polysaccharide conjugate may replace a lysine, arginine, or phenylalanine. In some embodiments, one or more nnAA incorporated into the polypeptide component of a polysaccharide conjugate may replace a lysine.

In some embodiments, the polypeptide component is a carrier protein. For instance, the polypeptide component of the polypeptide-polysaccharide conjugates described herein may be derived from CRM197 (SEQ ID NO: 1). In some embodiments, the polypeptide component of the polypeptide-polysaccharide conjugate comprises or consists of an amino acid sequence that is at least 80% identical to the amino acid sequence of SEQ ID NO: 1. In some embodiments, the polypeptide component of the polypeptide-polysaccharide conjugate comprises or consists of an amino acid sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to the amino acid sequence of SEQ ID NO: 1.

Unless otherwise indicated, percent identity is determined herein using the BLAST algorithm available on the World Wide Web at the following address:

blast.ncbi.nlm.nih.gov/Blast.cgi.

In some embodiments, the polypeptide component of the polypeptide-polysaccharide conjugate comprises or consists of the amino acid sequence of SEQ ID NO: 2. In certain embodiments, the polypeptide component of the polypeptide-polysaccharide conjugate comprises or consists of an amino acid sequence that is at least 90% or at least 95% identical to the amino acid sequence of SEQ ID NO: 2. In some embodiments, the polypeptide component of the polypeptide-polysaccharide conjugate comprises or consists of an amino acid sequence that is at least 90%, 91%, 92%, 93%, 94%, 95%, 96%, 97%, 98%, or 99% identical to the amino acid sequence of SEQ ID NO: 2. In some embodiments, the polypeptide component of the polypeptide-polysaccharide conjugate comprises or consists of an amino acid sequence that is at least 90%, 91%, 92%, 93%, 94%, 95%, 96%, 97%, 98%, or 99% identical to the amino acid sequence of SEQ ID NO: 2, where the nnAA are present in the positions specified in SEQ ID NO:2 and said sequence does not comprise an Arg-Arg dipeptide sequence. In certain embodiments, it should be understood that polypeptide components comprising or consisting of an amino acid sequence that is at least 90% or at least 95% identical to the amino acid sequence of SEQ ID NO: 2 maintain residues R193 and N194 of SEQ ID NO: 2.

Polypeptide-polysaccharide conjugates described herein may, in some instances, comprise a substituted Group A Streptococcus (GAS) polypeptide antigen component. Examples of polypeptide antigens that are GAS polypeptide antigens or derived from GAS polypeptide antigens include C5a peptidase (UniProt P15926), streptolysin O (SLO, UniProt POC0I3), streptococcal immunoglobulin-binding protein 35 (Sib35, UniProt Q1XG74), Fibronectin binding protein F1 (Sfbl, UniProt Q48VN7), and Adhesion and Division polypeptide (SpyAD, UniProt Q9A1H3). In embodiments comprising a substituted GAS polypeptide antigen component in the polypeptide-polysaccharide conjugate, such antigens will comprise at least two nnAAs as described herein. The nnAA may be inserted into the sequence or be substituted for (i.e., replace) an amino acid present in the sequence (e.g., one or more lysine, arginine, or phenylalanine residues). In some embodiments, the nnAAs are substituted for lysine residues.

Polypeptide-polysaccharide conjugates described herein may, in some instances, comprise a substituted Shigella polypeptide antigen (e.g., IpaB) component. Examples of suitable substituted Shigella polypeptide antigens may be found in International PCT Publication No. WO2020205584A1, incorporated herein by reference in its entirety. In embodiments comprising a substituted Shigella polypeptide antigen component in the polypeptide-polysaccharide conjugate, such antigens will comprise at least two nnAAs as described herein. The nnAA may be inserted into the sequence or be substituted for (i.e., replace) an amino acid present in the sequence (e.g., one or more lysine, arginine, or phenylalanine residues). In some embodiments, the nnAAs are substituted for lysine residues.

The protective effects of the stabilized vaccine compositions described herein may be induced, in part, from the at least one polysaccharide component (e.g., an antigen) of the polypeptide-polysaccharide conjugates. The polysaccharide component of the at least one polypeptide-polysaccharide conjugate may be a capsular polysaccharide (e.g., from a bacterial cell). In some embodiments, the capsular polysaccharide is a capsular polysaccharide of Streptococcus pneumoniae, Neisseria meningitidis, Haemophilus influenzae, or Streptococcus pyogenes. In some embodiments, the capsular polysaccharide is a capsular polysaccharide of Streptococcus pneumoniae. In some embodiments, the polysaccharide component of the at least one polypeptide-polysaccharide conjugate may be a cell wall polysaccharide (e.g., from a bacterial cell). In some embodiments, the cell wall polysaccharide is a cell wall polysaccharide of Streptococcus pyogenes (e.g., group A carbohydrate). In some embodiments, the cell wall polysaccharide is a cell wall polysaccharide of Shigella (e.g., 0-antigen Shigella polysaccharide (OPS)).

In some embodiments the capsular polysaccharide comprises a capsular polysaccharide derived from Streptococcus pneumoniae. Streptococcus pneumoniae is an encapsulated Gram-positive bacterium that can cause pneumonia, bacteremia, and meningitis. There are 90 distinct documented serotypes of S. pneumoniae (outlined in e.g. Kalin, M. Thorax 1998:53:159-162) which bear capsular polysaccharides with serotype-specific repeating unit structures. Therefore, in some cases the antigen is a Streptococcus pneumoniae capsular polysaccharide selected from 1, 2, 3, 4, 5, 6A, 6B, 7F, 7A, 7B, 7C, 8, 9A, 9L, 9N, 9V, 1° F., 10A, 10B, 10C, 11F, 11A, 11B, 11C, I1D, 12F, 12A, 12B, 13, 14, 15F, 15A, 15B, 15C, 16F, 16A, 17F, 17A, 18F, 18A, 18B, 18C, 19F, 19A, 19B, 19C, 20A, 20B, 21, 22F, 22A, 23F, 23A, 23B, 24F, 24A, 24B, 25F, 25A, 27, 28F, 28A, 29, 31, 32F, 32A, 33F, 33A, 33B, 33C, 33D, 34, 35F, 35A, 35B, 35C, 36, 37, 38, 39, 40, 41F, 41A, 42, 43, 44, 45, 46, 47F, 47A, and 48 (Henrichsen J Clin Microbiol 1995; 33:2759-2762).

Because polyvalent vaccines (e.g., more than one polysaccharide serotype) may provide enhanced protection in a subject, in some embodiments, the at least one polypeptide-polysaccharide conjugates of the stabilized vaccine compositions described herein may comprise more than one distinct polypeptide-polysaccharide conjugates. For instance, the at least one polypeptide-polysaccharide may comprise at least 5, at least 10, at least 15, at least 20, at least 24, at least 25, at least 30, at least 31, or at least 35 distinct polypeptide-polysaccharide conjugates.

In some embodiments, the at least one polypeptide-polysaccharide conjugate comprises at least 20 distinct polypeptide-polysaccharide conjugates. In some embodiments, the at least 20 distinct polypeptide-polysaccharide conjugates comprise at least 24 distinct polypeptide-polysaccharide conjugates; wherein there is a distinct polypeptide-polysaccharide conjugate comprising a capsular polysaccharide for each of Streptococcus pneumoniae serotypes 1, 2, 3, 4, 5, 6A, 6B, 7F, 8, 9V, 9N, 10A, 11A, 12F, 14, 15B, 17F, 18C, 19A, 19F, 20B, 22F, 23F, and 33F. In some embodiments, the at least 20 distinct polypeptide-polysaccharide conjugates comprise at least 31 distinct polypeptide-polysaccharide conjugates; wherein there is a distinct polypeptide-polysaccharide conjugate comprising a capsular polysaccharide for each of Streptococcus pneumoniae serotypes 1, 2, 3, 4, 5, 6A, 6B, 7C, 7F, 8, 9V, 9N, 10A, 11A, 12F, 14, 15A, 15B, 16F, 17F, 18C, 19A, 19F, 20B, 22F, 23A, 23B, 23F, 31, 33F, and 35B.

S. pneumoniae capsular saccharides may be obtained directly from bacteria using isolation procedures known to one of ordinary skill in the art (see for example methods disclosed in U.S. Patent App. Pub. Nos. 2006/0228380, 2006/0228381, 2007/0184071, 2007/0184072, 2007/0231340, and 2008/0102498 and WO 2008/118752). They may also be obtained from a commercial source (e.g., ATCC).

In addition to Streptococcus pneumoniae polysaccharides, in some embodiments, the stabilized vaccine compositions described herein may comprise Group A Streptococcus (GAS) polysaccharides from S. pyogenes, such as the group A carbohydrate (GAC). S. pyogenes is a gram-positive bacterium responsible for a wide array of infections in humans, including pharyngitis, tonsillitis, scarlet fever, cellulitis, erysipelas, rheumatic fever, post-streptococcal glomerulonephritis, necrotizing fasciitis, myonecrosis and lymphangitis. The GAC is composed of a polyrhamnose backbone with an immunodominant GlcNAc side chain and is present on the surface of strains of all GAS serotypes irrespective of M type. In some embodiments, the conjugate polysaccharide is a variant of the GAC that lacks the immunodominant GlcNAc side chain (See e.g., International PCT Publication No. WO 2013/020090, U.S. Pat. No. 10,780,155, and Gao, N. J. et al., Dec. 29, 2020. Infectious Microbes and Diseases, doi: 10.1097/IM9.0000000000000044, which are incorporated by reference in their entirety). In some embodiments, the GAS polysaccharide is purified from GAS bacterial cultures, bacterial stocks, or host cells. In some embodiments, the GAS polysaccharide is a synthesized polysaccharide. Methods of polysaccharide synthesis are known in the art, see e.g., Zhao, et al., Org. Chem. Front., 2019, 6, 3589-3596. Suitable GAS polysaccharides and their production are described in PCT/US2021/018402, incorporated by reference herein in its entirety.

The stabilized vaccine compositions described herein may comprise Shigella (e.g., S. dysenteriae, S. flexneri, S. boydii, Sonnei) polysaccharides. Shigellosis, or Shigella dysentery, is caused by the invasion of colonic epithelial cells by Shigella bacteria. Shigella dysentery is a significant contributor to infant mortality in many regions of the world, and also causes outbreaks among aid workers and other travelers. Over 40 Shigella serotypes are known, classified based on O antigen polysaccharide diversity. S. flexneri and S. dysentery are believed to be the agents primarily responsible for endemic and epidemic dysentery (Arabshahi et al. (2018) Bioengineered 9(1):170-177).

As described above, polysaccharide components described herein may comprise a click chemistry reactive group and can therefore be conjugated to polypeptide components by means of a metal-free “click” reaction. Examples of metal-free click chemistry reactive groups and derivatization of polysaccharides can be found, for instance, in International PCT Publication No. WO 2018/126229, incorporated by reference herein in its entirety. For example, in some embodiments, the polysaccharides are modified with DBCO or DBCO-PEG (e.g., DBCO-PEG-NH₂). In some embodiments, the conjugate polysaccharides are modified with DBCO-(PEG)₄-NH₂.

In some embodiments, the capsular polysaccharide or cell wall polysaccharide has an average molecular weight of about 10 kDa to about 40 kDa; about 10 kDa to about 35 kDa; about 10 kDa to about 30 kDa; about 10 kDa to about 25 kDa; about 10 kDa to about 20 kDa; about 10 kDa to about 15 kDa; 15 kDa to about 40 kDa; about 15 kDa to about 35 kDa; about 15 kDa to about 30 kDa; about 15 kDa to about 25 kDa; about 15 kDa to about 20 kDa; 20 kDa to about 40 kDa; about 20 kDa to about 35 kDa; about 20 kDa to about 30 kDa; about 20 kDa to about 25 kDa; 25 kDa to about 40 kDa; about 25 kDa to about 35 kDa; about 25 kDa to about 30 kDa; about 30 kDa to about 40 kDa; about 30 kDa to about 35 kDa; or about 35 kDa to about 40 kDa. In some embodiments, the capsular polysaccharide or cell wall polysaccharide has an average molecular weight of about 10 kDa, about 15 kDa, about 20 kDa, about 25 kDa, about 30 kDa, about 35 kDa, or about 40 kDa.

In some embodiments, the polypeptide-polysaccharide conjugate has an average molecular mass of about 10 kDa to about 5000 kDa. In some embodiments, the polypeptide-polysaccharide conjugate has an average molecular mass of about 10 kDa to about 50 kDa. In some embodiments, the polypeptide-polysaccharide conjugate has an average molecular mass of about 10 kDa to about 100 kDa. In some embodiments, the polypeptide-polysaccharide conjugate has an average molecular mass of about 10 kDa to about 200 kDa. In some embodiments, the polypeptide-polysaccharide conjugate has an average molecular mass of about 10 kDa to about 500 kDa. In some embodiments, the polypeptide-polysaccharide conjugate has an average molecular mass of about 10 kDa to about 1000 kDa. In some embodiments, the polypeptide-polysaccharide conjugate has an average molecular mass of about 100 kDa to about 200 kDa. In some embodiments, the polypeptide-polysaccharide conjugate has an average molecular mass of about 100 kDa to about 300 kDa. In some embodiments, the polypeptide-polysaccharide conjugate has an average molecular mass of about 100 kDa to about 400 kDa. In some embodiments, the polypeptide-polysaccharide conjugate has an average molecular mass of about 100 kDa to about 500 kDa. In some embodiments, the polypeptide-polysaccharide conjugate has an average molecular mass of about 100 kDa to about 5000 kDa. In some embodiments, the polypeptide-polysaccharide conjugate has an average molecular mass of about 500 kDa to about 5000 kDa. In some embodiments, the polypeptide-polysaccharide conjugate has an average molecular mass of about 1000 kDa to about 5000 kDa. In some embodiments, the polypeptide-polysaccharide conjugate has an average molecular mass of about 2000 kDa to about 5000 kDa. In some embodiments, the polypeptide-polysaccharide conjugate has an average molecular mass of about 3000 kDa to about 5000 kDa. In some embodiments, the polypeptide-polysaccharide conjugate has an average molecular mass of about 4000 kDa to about 5000 kDa. In some embodiments, the polypeptide-polysaccharide conjugate has an average molecular mass of about 1000 kDa to about 3000 kDa. In some embodiments, the polypeptide-polysaccharide conjugate has an average molecular mass of about 1000 kDa to about 2000 kDa. In some embodiments, the polypeptide-polysaccharide conjugate has an average molecular mass of about 2000 kDa to about 3000 kDa. In some embodiments, the polypeptide-polysaccharide conjugate has an average molecular mass of about 5000 kDa or greater.

### Aluminum Adjuvants and Non-Aluminum Phosphate Salts

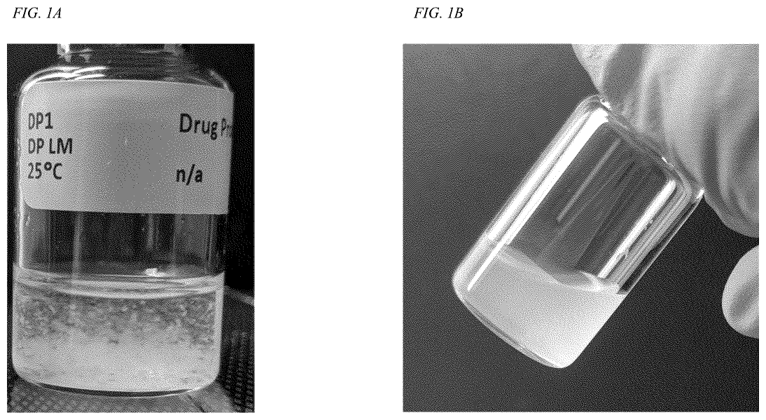

Aluminum adjuvants are often used in vaccines to enhance the ability of an antigen to promote an immune response, improve the potency of the vaccine, or reduce the frequency of vaccination and/or dose of antigen per vaccination. However, as described earlier, aluminum adjuvants may flocculate or form aggregates upon resuspension of a drug product, rendering the vaccine unsuitable for administration. The stabilized vaccine compositions described herein address this issue by incorporating non-aluminum phosphate salts, which alter the resuspension characteristics such that a homogenous suspension of the adjuvant and/or adjuvant-conjugate complex in the drug product is achieved. FIG. 1A shows a flocculated vaccine composition unsuitable for administration and FIG. 1B shows an exemplary resuspended stabilized vaccine composition described herein.

The stabilized vaccine compositions described herein comprise an aluminum adjuvant. In some embodiments, the aluminum adjuvant is aluminum phosphate (ALPO), aluminum hydroxide (ALOH), amorphous aluminum hydroxyphosphate sulfate, or aluminum potassium phosphate. In some embodiments, the aluminum adjuvant is ALPO.

The concentration of the aluminum adjuvant may be any concentration suitable for use in a mammalian vaccine product, for instance, any concentration approved for use by a drug regulatory agency. In some embodiments, the concentration of the aluminum adjuvant is between about 125 μg/mL and about 250 μg/mL; between about 125 μg/mL and about 225 μg/mL; between about 125 μg/mL and about 200 μg/mL; between about 125 μg/mL and about 175 μg/mL; between about 125 μg/mL and about 150 μg/mL; between about 150 μg/mL and about 250 μg/mL; between about 150 μg/mL and about 225 μg/mL; between about 150 μg/mL and about 200 μg/mL; between about 150 μg/mL and about 175 μg/mL; between about 175 μg/mL and about 250 μg/mL; between about 175 μg/mL and about 225 μg/mL; between about 175 μg/mL and about 200 μg/mL; between about 200 μg/mL and about 250 μg/mL; and between about 200 μg/mL and about 225 μg/mL. In some embodiments, the concentration of the aluminum adjuvant is about 125 μg/mL, about 150 μg/mL, about 175 μg/mL, about 200 μg/mL, about 225 μg/mL, or about 250 μg/mL.

The use of non-aluminum phosphate salts to the stabilized vaccine compositions described herein may result in reduction of adjuvant flocculation and aggregation. The non-aluminum phosphate salt may be, for instance, sodium phosphate or potassium phosphate. In some embodiments, the concentration of the non-aluminum phosphate salt is between about 15 mM and about 20 mM. In certain embodiments, the concentration of the non-aluminum phosphate salt is between about 15 mM and about 19 mM; between about 15 mM and about 18 mM; between about 15 mM and about 17 mM; between about 15 mM and about 16 mM; between about 16 mM and about 20 mM; between about 16 mM and about 19 mM; between about 16 mM and about 18 mM; between about 16 mM and about 17 mM; between about 17 mM and about 20 mM; between about 17 mM and about 19 mM; between about 17 mM and about 18 mM; between about 18 mM and about 20 mM; between about 18 mM and about 19 mM; or between about 19 mM and about 20 mM. In certain embodiments, the concentration of the non-aluminum phosphate salt is about 15 mM, about 16 mM, about 17 mM, about 18 mM, about 19 mM, or about 20 mM.

Stabilized vaccine compositions may also comprise sodium chloride, although the concentrations are much lower than would otherwise be required to prevent flocculation in similar non-stabilized compositions. This keeps the osmolality of the vaccine at a level that prevents potential undesirable pain at the site of injection in a subject. In some embodiments, the concentration of sodium chloride is less than about 200 mM. In certain embodiments, the concentration of sodium chloride is between about 1 mM and less than about 200 mM. In certain embodiments, the concentration of sodium chloride is between about 1 mM and about 175 mM; between about 1 mM and about 150 mM; between about 1 mM and about 125 mM; between about 1 mM and about 100 mM; between about 1 mM and about 75 mM; between about 1 mM and about 50 mM; between about 1 mM and about 25 mM; between about 25 mM and about less than about 200 mM; between about 25 mM and about 175 mM; between about 25 mM and about 150 mM; between about 25 mM and about 125 mM; between about 25 mM and about 100 mM; between about 25 mM and about 75 mM; between about 25 mM and about 50 mM; between about 50 mM and about less than about 200 mM; between about 50 mM and about 175 mM; between about 50 mM and about 150 mM; between about 50 mM and about 125 mM; between about 50 mM and about 100 mM; between about 50 mM and about 75 mM; between about 75 mM and about less than about 200 mM; between about 75 mM and about 175 mM; between about 75 mM and about 150 mM; between about 75 mM and about 125 mM; between about 75 mM and about 100 mM; between about 100 mM and about less than about 200 mM; between about 100 mM and about 175 mM; between about 100 mM and about 150 mM; between about 100 mM and about 125 mM; between about 125 mM and about less than about 200 mM; between about 125 mM and about 175 mM; between about 125 mM and about 150 mM; between about 150 mM and about less than about 200 mM; between about 150 mM and about 175 mM; or between about 175 mM and about less than about 200 mM. In some embodiments, the concentration of sodium chloride is about 1 mM, about 25 mM, about 50 mM, about 75 mM, about 100 mM, about 125 mM, about 150 mM, about 175 mM, or about less than about 200 mM.

Stabilized vaccine compositions may comprise an emulsifier. In some embodiments, the emulsifier is a polysorbate. In some embodiments, the emulsifier is polysorbate 20, polysorbate 40, polysorbate 60, or polysorbate 80. In some embodiments, the concentration of the emulsifier is between about 0.01% and about 5.0%; between about 0.01% and about 4.0%; between about 0.01% and about 3.0%; between about 0.01% and about 2.0%; between about 0.01% and about 1.0%; between about 0.01% and about 0.5%; between about 0.01% and about 0.1%; between about 0.01% and about 0.05%; between about 0.05% and about 5.0%; between about 0.05% and about 4.0%; between about 0.05% and about 3.0%; between about 0.05% and about 2.0%; between about 0.05% and about 1.0%; between about 0.05% and about 0.5%; between about 0.05% and about 0.1%; between about 0.1% and about 5.0%; between about 0.1% and about 4.0%; between about 0.1% and about 3.0%; between about 0.1% and about 2.0%; between about 0.1% and about 1.0%; between about 0.1% and about 0.5%; between about 0.5% and about 5.0%; between about 0.5% and about 4.0%; between about 0.5% and about 3.0%; between about 0.5% and about 2.0%; between about 0.5% and about 1.0%; between about 1.0% and about 5.0%; between about 1.0% and about 4.0%; between about 1.0% and about 3.0%; between about 1.0% and about 2.0%; between about 2.0% and about 5.0%; between about 2.0% and about 4.0%; between about 2.0% and about 3.0%; between about 3.0% and about 5.0%; between about 3.0% and about 4.0%; and between about 4.0% and about 5.0%.

The non-aluminum phosphate salt of the presently described stabilized vaccine compositions enables resuspension of the aluminum adjuvant without drastically modulating the pH of the composition. It has been shown that elevated pH may result in degradation of certain polysaccharides (see, for example: Pujar, N. S., Biopolymers, 2004, 75(1):71-84). In some embodiments, the pH of the stabilized vaccine composition is between about 5.5 and about 7; between about 5.5 and about 6.9; between about 5.5 and about 6.8; between about 5.5 and about 6.7; between about 5.5 and about 6.6; between about 5.5 and about 6.5; between about 5.5 and about 6.4; between about 5.5 and about 6.3; between about 5.5 and about 6.2; between about 5.5 and about 6.1; between about 5.5 and about 6.0; between about 5.5 and about 5.9; between about 5.5 and about 5.8; between about 5.5 and about 5.7; between about 5.6 and about 7; between about 5.6 and about 6.9; between about 5.6 and about 6.8; between about 5.6 and about 6.7; between about 5.6 and about 6.6; between about 5.6 and about 6.5; between about 5.6 and about 6.4; between about 5.6 and about 6.3; between about 5.6 and about 6.2; between about 5.6 and about 6.1; between about 5.6 and about 6.0; between about 5.6 and about 5.9; between about 5.6 and about 5.8; between about 5.6 and about 5.7; between about 5.7 and about 7; between about 5.7 and about 6.9; between about 5.7 and about 6.8; between about 5.7 and about 6.7; between about 5.7 and about 6.6; between about 5.7 and about 6.5; between about 5.7 and about 6.4; between about 5.7 and about 6.3; between about 5.7 and about 6.2; between about 5.7 and about 6.1; between about 5.7 and about 6.0; between about 5.7 and about 5.9; between about 5.7 and about 5.8; between about 5.8 and about 7; between about 5.8 and about 6.9; between about 5.8 and about 6.8; between about 5.8 and about 6.7; between about 5.8 and about 6.6; between about 5.8 and about 6.5; between about 5.8 and about 6.4; between about 5.8 and about 6.3; between about 5.8 and about 6.2; between about 5.8 and about 6.1; between about 5.8 and about 6.0; between about 5.8 and about 5.9; between about 5.9 and about 7; between about 5.9 and about 6.9; between about 5.9 and about 6.8; between about 5.9 and about 6.7; between about 5.9 and about 6.6; between about 5.9 and about 6.5; between about 5.9 and about 6.4; between about 5.9 and about 6.3; between about 5.9 and about 6.2; between about 5.9 and about 6.1; between about 6.0 and about 7.0; between about 6.0 and about 6.9; between about 6.0 and about 6.8; between about 6.0 and about 6.7; between about 6.0 and about 6.6; between about 6.0 and about 6.5; between about 6.0 and about 6.4; between about 6.0 and about 6.3; between about 6.0 and about 6.2; between about 6.0 and about 6.1; between about 6.1 and about 7.0; between about 6.1 and about 6.9; between about 6.1 and about 6.8; between about 6.1 and about 6.7; between about 6.1 and about 6.6; between about 6.1 and about 6.5; between about 6.1 and about 6.4; between about 6.1 and about 6.3; between about 6.1 and about 6.2; between about 6.2 and about 7.0; between about 6.2 and about 6.9; between about 6.2 and about 6.8; between about 6.2 and about 6.7; between about 6.2 and about 6.6; between about 6.2 and about 6.5; between about 6.2 and about 6.4; between about 6.2 and about 6.3; between about 6.3 and about 7.0; between about 6.3 and about 6.9; between about 6.3 and about 6.8; between about 6.3 and about 6.7; between about 6.3 and about 6.6; between about 6.3 and about 6.5; between about 6.3 and about 6.4; between about 6.4 and about 7.0; between about 6.4 and about 6.9; between about 6.4 and about 6.8; between about 6.4 and about 6.7; between about 6.4 and about 6.6; between about 6.4 and about 6.5; between about 6.5 and about 7.0; between about 6.5 and about 6.9; between about 6.5 and about 6.8; between about 6.5 and about 6.7; between about 6.5 and about 6.6; between about 6.6 and about 7.0; between about 6.6 and about 6.9; between about 6.6 and about 6.8; between about 6.6 and about 6.7; between about 6.7 and about 7.0; between about 6.7 and about 6.9; between about 6.7 and about 6.8; between about 6.8 and about 7.0; between about 6.8 and about 6.9; or between about 6.9 and about 7.0. In certain embodiments, the pH of the stabilized vaccine composition is about 5.5, about 5.6, about 5.7, about 5.8, about 5.9, about 6.0, about 61, about 6.2, about 6.3, about 6.4, about 6.5, about 6.6, about 6.7, about 6.8, about 6.9, or about 7.0.

As described above, stabilized vaccine compositions comprising a non-aluminum phosphate salt prevent flocculation of adjuvant or adjuvant-conjugate complexes. Resultingly, the vaccine compositions may be homogenous. The stabilized vaccine compositions described herein may comprise a liquid solution and an alum suspension. It may be useful to characterize the alum suspension in terms of physical properties that are differentiable from those of aggregates and/or flocculants unsuitable for administration to a subject.

An alum suspension may, for instance, be characterized by particle size. In some embodiments, the alum suspension does not comprise particles greater than about 100 μm, about 200 μm, about 300 μm, or about 400 μm as measured by a suitable analytical technique (e.g., light scattering). The alum suspension may also be characterized by values such as D10, D50, and D90, which describe the portion of particles (e.g., 10%, 50%, and 90%) with diameters below the given value. In some embodiments, the alum suspension comprises or consists of particles with a D50 value of between about 1 μm and about 10 μm; between about 1 μm and about 9 μm; between about 1 μm and about 8 μm; between about 1 μm and about 7 μm; between about 1 μm and about 6 μm; between about 1 μm and about 5 μm; between about 1 μm and about 4 μm; between about 1 μm and about 3 μm; between about 1 μm and about 2 μm; between about 2 μm and about 10 μm; between about 2 μm and about 9 μm; between about 2 μm and about 8 μm; between about 2 μm and about 7 μm; between about 2 μm and about 6 μm; between about 2 μm and about 5 μm; between about 2 μm and about 4 μm; between about 2 μm and about 3 μm; between about 3 μm and about 10 μm; between about 3 μm and about 9 μm; between about 3 μm and about 8 μm; between about 2 μm and about 7 μm; between about 3 μm and about 6 μm; between about 3 μm and about 5 μm; between about 3 μm and about 4 μm; between about 4 μm and about 10 μm; between about 4 μm and about 9 μm; between about 4 μm and about 8 μm; between about 4 μm and about 7 μm; between about 4 μm and about 6 μm; between about 4 μm and about 5 μm; between about 5 μm and about 10 μm; between about 5 μm and about 9 μm; between about 5 μm and about 8 μm; between about 5 μm and about 7 μm; between about 5 μm and about 6 μm; between about 6 μm and about 10 μm; between about 6 μm and about 9 μm; between about 6 μm and about 8 μm; between about 6 μm and about 7 μm; between about 7 μm and about 10 μm; between about 7 μm and about 9 μm; between about 7 μm and about 8 μm; between about 8 μm and about 10 μm; between about 8 μm and about 9 μm; or between about 9 μm and about 10 μm. In some embodiments, the alum suspension comprises or consists of particles with a D50 value of about 1 μm, about 2 μm, about 3 μm, about 4 μm, about 5 μm, about 6 μm, about 7 μm, about 8 μm, about 9 μm, or about 10 μm.

### Additional Unsubstituted Polypeptide Antigens

The stabilized vaccine compositions described herein may further comprise or consist of at least one unsubstituted polypeptide antigen. For example, in some embodiments, when the polysaccharide component of the polypeptide-polysaccharide conjugate is a GAS polysaccharide, the stabilized vaccine composition may further comprise a C5a unsubstituted polypeptide antigen and/or a SLO unsubstituted polypeptide antigen. In some embodiments, the at least one unsubstituted polypeptide antigen comprises a Group A Streptococcus (GAS) unsubstituted polypeptide antigen (e.g., C5a, SLO, Sib35, Sfbl or a combination of the foregoing).

In some embodiments, the at least one unsubstituted polypeptide antigen comprises or consists of a first Group A Streptococcus (GAS) unsubstituted polypeptide antigen and a second GAS unsubstituted polypeptide antigen. In some embodiments, the polysaccharide component of the at least one polypeptide-polysaccharide conjugate is a GAS polysaccharide or a variant thereof that lacks an immunodominant N-acetyl Glucosamine (GlcNAc) side chain.

### Exemplary Stabilized Vaccine Compositions

As described above, stabilized vaccine compositions may comprise at least one polypeptide-polysaccharide conjugate, wherein the polypeptide component of the at least one polypeptide-polysaccharide conjugate comprises at least two non-natural amino acids (nnAA); an aluminum adjuvant; a non-aluminum phosphate salt; and sodium chloride; wherein the concentration of sodium chloride is less than 200 mM, and the pH is between about 5.5 and 7.

In some embodiments, the stabilized vaccine composition comprises at least one polypeptide-polysaccharide conjugate, wherein the polypeptide component comprises or consists of an amino acid sequence that is at least 90%, 91%, 92%, 93%, 94%, 95%, 96%, 97%, 98%, or 99% identical to the amino acid sequence of SEQ ID NO: 2; about 250 μg/mL of an aluminum phosphate or aluminum hydroxide adjuvant; about 17 mM of potassium phosphate or sodium phosphate; and about 150 mM of sodium chloride; wherein the pH is about 5.8. In some embodiments, the polypeptide component of the polypeptide-polysaccharide conjugate comprises or consists of an amino acid sequence that is at least 90%, 91%, 92%, 93%, 94%, 95%, 96%, 97%, 98%, or 99% identical to the amino acid sequence of SEQ ID NO: 2, where the nnAA are present in the positions specified in SEQ ID NO: 2.

In some embodiments, the stabilized vaccine composition comprises at least 24 distinct polypeptide-polysaccharide conjugates, wherein the polypeptide component comprises or consists of an amino acid sequence that is at least 90%, 91%, 92%, 93%, 94%, 95%, 96%, 97%, 98%, or 99% identical to the amino acid sequence of SEQ ID NO: 2, and there is a distinct polypeptide-polysaccharide conjugate comprising a capsular polysaccharide for each of Streptococcus pneumoniae serotypes 1, 2, 3, 4, 5, 6A, 6B, 7F, 8, 9V, 9N, 10A, 11A, 12F, 14, 15B, 17F, 18C, 19A, 19F, 20B, 22F, 23F, and 33F; an aluminum adjuvant; a non-aluminum phosphate salt; and sodium chloride; wherein the concentration of sodium chloride is less than 200 mM, and the pH is between about 5.5 and 7. In some embodiments, the polypeptide component of the polypeptide-polysaccharide conjugate comprises or consists of an amino acid sequence that is at least 90%, 91%, 92%, 93%, 94%, 95%, 96%, 97%, 98%, or 99% identical to the amino acid sequence of SEQ ID NO: 2, where the nnAA are present in the positions specified in SEQ ID NO: 2.

In some embodiments, the stabilized vaccine composition comprises at least 24 distinct polypeptide-polysaccharide conjugates, wherein the polypeptide component comprises or consists of an amino acid sequence that is at least 90%, 91%, 92%, 93%, 94%, 95%, 96%, 97%, 98%, or 99% identical to the amino acid sequence of SEQ ID NO: 2, and there is a distinct polypeptide-polysaccharide conjugate comprising a capsular polysaccharide for each of Streptococcus pneumoniae serotypes 1, 2, 3, 4, 5, 6A, 6B, 7F, 8, 9V, 9N, 10A, 11A, 12F, 14, 15B, 17F, 18C, 19A, 19F, 20B, 22F, 23F, and 33F; about 250 μg/mL of an aluminum phosphate or aluminum hydroxide adjuvant; about 17 mM of potassium phosphate or sodium phosphate; and about 150 mM of sodium chloride; wherein the pH is about 5.8. In some embodiments, the polypeptide component of the polypeptide-polysaccharide conjugate comprises or consists of an amino acid sequence that is at least 90%, 91%, 92%, 93%, 94%, 95%, 96%, 97%, 98%, or 99% identical to the amino acid sequence of SEQ ID NO: 2, where the nnAA are present in the positions specified in SEQ ID NO: 2.

In some embodiments, the stabilized vaccine composition comprises at least 31 distinct polypeptide-polysaccharide conjugates, wherein the polypeptide component comprises or consists of an amino acid sequence that is at least 90%, 91%, 92%, 93%, 94%, 95%, 96%, 97%, 98%, or 99% identical to the amino acid sequence of SEQ ID NO: 2, and there is a distinct polypeptide-polysaccharide conjugate comprising a capsular polysaccharide for each of Streptococcus pneumoniae serotypes 1, 2, 3, 4, 5, 6A, 6B, 7C, 7F, 8, 9V, 9N, 10A, 11A, 12F, 14, 15A, 15B, 16F, 17F, 18C, 19A, 19F, 20B, 22F, 23A, 23B, 23F, 31, 33F, and 35B; an aluminum adjuvant; a non-aluminum phosphate salt; and sodium chloride; wherein the concentration of sodium chloride is less than 200 mM, and the pH is between about 5.5 and 7. In some embodiments, the polypeptide component of the polypeptide-polysaccharide conjugate comprises or consists of an amino acid sequence that is at least 90%, 91%, 92%, 93%, 94%, 95%, 96%, 97%, 98%, or 99% identical to the amino acid sequence of SEQ ID NO: 2, where the nnAA are present in the positions specified in SEQ ID NO: 2.

In some embodiments, the stabilized vaccine composition comprises at least 31 distinct polypeptide-polysaccharide conjugates, wherein the polypeptide component comprises or consists of an amino acid sequence that is at least 90%, 91%, 92%, 93%, 94%, 95%, 96%, 97%, 98%, or 99% identical to the amino acid sequence of SEQ ID NO: 2, and there is a distinct polypeptide-polysaccharide conjugate comprising a capsular polysaccharide for each of Streptococcus pneumoniae serotypes 1, 2, 3, 4, 5, 6A, 6B, 7C, 7F, 8, 9V, 9N, 10A, 11A, 12F, 14, 15A, 15B, 16F, 17F, 18C, 19A, 19F, 20B, 22F, 23A, 23B, 23F, 31, 33F, and 35B; about 250 μg/mL of an aluminum phosphate or aluminum hydroxide adjuvant; about 17 mM of potassium phosphate or sodium phosphate; and about 150 mM of sodium chloride; wherein the pH is about 5.8. In some embodiments, the polypeptide component of the polypeptide-polysaccharide conjugate comprises or consists of an amino acid sequence that is at least 90%, 91%, 92%, 93%, 94%, 95%, 96%, 97%, 98%, or 99% identical to the amino acid sequence of SEQ ID NO: 2, where the nnAA are present in the positions specified in SEQ ID NO: 2.

In some embodiments, the stabilized vaccine composition further comprises at least one unsubstituted polypeptide antigen. In some instances, the at least one unsubstituted polypeptide antigen comprises a first Group A Streptococcus (GAS) unsubstituted polypeptide antigen, and a second GAS unsubstituted polypeptide antigen, and wherein the polysaccharide component of the at least one polypeptide-polysaccharide conjugate is a GAS polysaccharide or a variant thereof that lacks an immunodominant N-acetyl Glucosamine (GlcNAc) side chain.

### Therapeutic Methods and Uses

Also included herein are methods of inducing a protective immune response against an infectious pathogen in a subject, comprising administering a stabilized vaccine composition described herein to a subject. In some embodiments, the infectious pathogen may be, for example, S. pneumoniae, S. pyogenes (Group A Streptococcus), Neisseria meningitidis, Haemophilus influenzae, S. dysenteriae, S. flexneri, S. boydii, or S. sonnei.

Also described herein are methods for immunizing a subject against pneumococcal infection. In some embodiments, a method for immunizing a subject against pneumococcal infection comprises administering a stabilized vaccine composition described herein. In some embodiments, the stabilized vaccine composition comprises at least 24 distinct polypeptide-polysaccharide conjugates, wherein the polypeptide component comprises or consists of an amino acid sequence that is at least 90%, 91%, 92%, 93%, 94%, 95%, 96%, 97%, 98%, or 99% identical to the amino acid sequence of SEQ ID NO: 2, and there is a distinct polypeptide-polysaccharide conjugate comprising a capsular polysaccharide for each of Streptococcus pneumoniae serotypes 1, 2, 3, 4, 5, 6A, 6B, 7F, 8, 9V, 9N, 10A, 11A, 12F, 14, 15B, 17F, 18C, 19A, 19F, 20B, 22F, 23F, and 33F; an aluminum adjuvant; a non-aluminum phosphate salt; and sodium chloride; wherein the concentration of sodium chloride is less than 200 mM, and the pH is between about 5.5 and 7. In some embodiments, the vaccine composition comprises about 250 μg/mL of an aluminum phosphate or aluminum hydroxide adjuvant; about 17 mM of potassium phosphate or sodium phosphate; and about 150 mM of sodium chloride; wherein the pH is about 5.8. In some embodiments, the polypeptide component of the polypeptide-polysaccharide conjugate comprises or consists of an amino acid sequence that is at least 90%, 91%, 92%, 93%, 94%, 95%, 96%, 97%, 98%, or 99% identical to the amino acid sequence of SEQ ID NO: 2, where the nnAA are present in the positions specified in SEQ ID NO: 2.

In certain embodiments of the methods for immunizing a subject against pneumococcal infection, the method comprises administering a stabilized vaccine composition comprising at least 31 distinct polypeptide-polysaccharide conjugates, wherein the polypeptide component comprises or consists of an amino acid sequence that is at least 90%, 91%, 92%, 93%, 94%, 95%, 96%, 97%, 98%, or 99% identical to the amino acid sequence of SEQ ID NO: 2, and there is a distinct polypeptide-polysaccharide conjugate comprising a capsular polysaccharide for each of Streptococcus pneumoniae serotypes 1, 2, 3, 4, 5, 6A, 6B, 7C, 7F, 8, 9V, 9N, 10A, 11A, 12F, 14, 15A, 15B, 16F, 17F, 18C, 19A, 19F, 20B, 22F, 23A, 23B, 23F, 31, 33F, and 35B; an aluminum adjuvant; a non-aluminum phosphate salt; and sodium chloride; wherein the concentration of sodium chloride is less than 200 mM, and the pH is between about 5.5 and 7. In some embodiments, the vaccine composition comprises about 250 μg/mL of an aluminum phosphate or aluminum hydroxide adjuvant; about 17 mM of potassium phosphate or sodium phosphate; and about 150 mM of sodium chloride; wherein the pH is about 5.8. In some embodiments, the polypeptide component of the polypeptide-polysaccharide conjugate comprises or consists of an amino acid sequence that is at least 90%, 91%, 92%, 93%, 94%, 95%, 96%, 97%, 98%, or 99% identical to the amino acid sequence of SEQ ID NO: 2, where the nnAA are present in the positions specified in SEQ ID NO: 2.

Also included herein is the use of a stabilized vaccine composition described herein in the manufacture of a medicament for inducing a protective immune response against S. pneumoniae, S. pyogenes (Group A Streptococcus), Neisseria meningitidis, Haemophilus influenzae, S. dysenteriae, S. flexneri, S. boydii, or S. sonnei in a subject. In some embodiments is the use of a stabilized vaccine composition described herein in the manufacture of a medicament for inducing a protective immune response against S. pneumoniae, S. pyogenes (Group A Streptococcus). Also provided are the use of stabilized vaccine compositions described herein in the manufacture of a medicament for inducing a protective immune response against pneumococcal infection in a subject.

Also included herein are stabilized vaccine compositions described herein for use in inducing a protective immune response against an infectious pathogen in a subject. In some embodiments of a stabilized vaccine composition for use in inducing a protective immune response against an infectious pathogen in a subject, the infectious pathogen may be S. pneumoniae, S. pyogenes (Group A Streptococcus), Neisseria meningitidis, Haemophilus influenzae, S. dysenteriae, S. flexneri, S. boydii, or S. sonnei. In some embodiments, the infectious pathogen may be S. pneumoniae or S. pyogenes (Group A Streptococcus).

Also provided are methods of stabilizing a vaccine composition comprising mixing a non-aluminum phosphate salt with a solution comprising at least one polypeptide-polysaccharide conjugate, wherein the polypeptide component of the at least one polypeptide-polysaccharide conjugate comprises at least two non-natural amino acids (nnAAs); an aluminum adjuvant; and sodium chloride; wherein the concentration of sodium chloride is less than 200 mM, and the pH is between about 5.5 and 7. In some embodiments of a method of stabilizing a vaccine composition, the vaccine composition comprises about 250 μg/mL of an aluminum phosphate or aluminum hydroxide adjuvant; about 17 mM of potassium phosphate or sodium phosphate; and about 150 mM of sodium chloride; wherein the pH is about 5.8.

### ENUMERATED EMBODIMENTS

Embodiment I-1. A stabilized vaccine composition comprising:
a. at least one polypeptide-polysaccharide conjugate, wherein the polypeptide component of the at least one polypeptide-polysaccharide conjugate comprises at least two non-natural amino acids (nnAA);
b. an aluminum adjuvant;
c. a non-aluminum phosphate salt; and
d. sodium chloride;
wherein the concentration of sodium chloride is less than 200 mM, and the pH is between about 5.5 and 7.

Embodiment I-2. The stabilized vaccine composition of embodiment I-1, further comprising an emulsifier.

Embodiment I-3. The stabilized vaccine composition of embodiment I-1 or I-2, wherein the polypeptide component of the at least one polypeptide-polysaccharide conjugate is a substituted polypeptide antigen.

Embodiment I-4. The stabilized vaccine composition of embodiment I-1 or I-2, wherein the polypeptide component of the at least one polypeptide-polysaccharide conjugate is a carrier protein.

Embodiment I-5. The stabilized vaccine composition of any one of embodiments I-1 to I-4, wherein the polysaccharide component of the at least one polypeptide-polysaccharide conjugate is a capsular polysaccharide.

Embodiment I-6. The stabilized vaccine composition of embodiment I-5, wherein the capsular polysaccharide is a capsular polysaccharide of Streptococcus pneumoniae, Neisseria meningitidis, Haemophilus influenzae, or Streptococcus pyogenes.

Embodiment I-7. The stabilized vaccine composition of embodiment I-5 or I-6, wherein the capsular polysaccharide is a capsular polysaccharide of Streptococcus pneumoniae.

Embodiment I-8. The stabilized vaccine composition of any one of embodiments I-1 to I-7, wherein the at least one polypeptide-polysaccharide conjugate comprises at least 20 distinct polypeptide-polysaccharide conjugates.

Embodiment I-9. The stabilized vaccine composition of embodiment I-8, wherein the at least 20 distinct polypeptide-polysaccharide conjugates comprise at least 24 distinct polypeptide-polysaccharide conjugates; wherein there is a distinct polypeptide-polysaccharide conjugate comprising a capsular polysaccharide for each of Streptococcus pneumoniae serotypes 1, 2, 3, 4, 5, 6A, 6B, 7F, 8, 9V, 9N, 10A, 11A, 12F, 14, 15B, 17F, 18C, 19A, 19F, 20B, 22F, 23F, and 33F.

Embodiment I-10. The stabilized vaccine composition of embodiment I-8, wherein the at least 20 distinct polypeptide-polysaccharide conjugates comprise at least 31 distinct polypeptide-polysaccharide conjugates; wherein there is a distinct polypeptide-polysaccharide conjugate comprising a capsular polysaccharide for each of Streptococcus pneumoniae serotypes 1, 2, 3, 4, 5, 6A, 6B, 7C, 7F, 8, 9V, 9N, 10A, 11A, 12F, 14, 15A, 15B, 16F, 17F, 18C, 19A, 19F, 20B, 22F, 23A, 23B, 23F, 31, 33F, and 35B.

Embodiment I-11. The stabilized vaccine composition of any one of embodiments I-1 to I-4, wherein the polysaccharide component of the at least one polypeptide-polysaccharide conjugate is a cell wall polysaccharide.

Embodiment I-12. The stabilized vaccine composition of any one of embodiments I-1 to I-4 or I-11, further comprising at least one unsubstituted polypeptide antigen.

Embodiment I-13. The stabilized vaccine composition of any one of embodiments I-1 to I-12, wherein the at least two nnAAs are selected from the group consisting of 2-amino-3-(4-azidophenyl)propanoic acid (pAF), 2-amino-4-azidobutanoic acid, 2-azido-3-phenylpropionic acid, 2-amino-3-azidopropanoic acid, 2-amino-3-(4-(azidomethyl)phenyl)propanoic acid (pAMF), 2-amino-3-(5-(azidomethyl)pyridin-2-yl)propanoic acid, 2-amino-3-(4-(azidomethyl)pyridin-2-yl)propanoic acid, 2-amino-3-(6-(azidomethyl)pyridin-3-yl)propanoic acid, and 2-amino-5-azidopentanoic acid.

Embodiment I-14. The stabilized vaccine composition of any one of embodiments I-1 to I-13, wherein the at least two nnAAs are pAMF.

Embodiment I-15. The stabilized vaccine composition of any one of embodiments I-1 to I-14, wherein the polypeptide component of the polypeptide-polysaccharide conjugate comprises 3-8 nnAA and the nnAA present are all the same nnAA.

Embodiment I-16. The stabilized vaccine composition of any one of embodiments I-1 to I-2, or I-4 to I-15 wherein the polypeptide component of the polypeptide-polysaccharide conjugate comprises an amino acid sequence that is at least 80% identical to SEQ ID NO: 1.

Embodiment I-17. The stabilized vaccine composition of any one of embodiments I-1 to I-2, or I-4 to I-15, wherein the polypeptide component of the polypeptide-polysaccharide conjugate comprises an amino acid sequence that is at least 90% or 95% identical to SEQ ID NO: 1.

Embodiment I-18. The stabilized vaccine composition of any one of embodiments I-1 to I-2, or I-4 to I-15, wherein the polypeptide component of the polypeptide-polysaccharide conjugate comprises or consists of the amino acid sequence of SEQ ID NO: 2.

Embodiment I-19. The stabilized vaccine composition of any one of embodiments I-1 to I-18, wherein the aluminum adjuvant is aluminum phosphate (ALPO), aluminum hydroxide (ALOH), amorphous aluminum hydroxyphosphate sulfate, or aluminum potassium phosphate.

Embodiment I-20. The stabilized vaccine composition of any one of embodiments I-1 to I-18, wherein the aluminum adjuvant is ALPO.

Embodiment I-21. The stabilized vaccine composition of any one of embodiments I-1 to I-20, wherein the concentration of the aluminum adjuvant is between about 125 μg/mL and about 250 μg/mL.

Embodiment I-22. The stabilized vaccine composition of any one of embodiments I-1 to I-22, wherein the concentration of the non-aluminum phosphate salt is between about 15 mM and 20 mM.

Embodiment I-23. The stabilized vaccine composition of any one of embodiments I-1 to I-22, wherein the concentration of the non-aluminum phosphate salt is about 17 mM.

Embodiment I-24. The stabilized vaccine composition of any one of embodiments I-1 to I-23, wherein the concentration of sodium chloride is about 150 mM.

Embodiment I-25. The stabilized vaccine composition of any one of embodiments I-1 to I-24, wherein the pH is about 5.8.

Embodiment I-26. The stabilized vaccine composition of any one of embodiments I-1 to I-25, wherein the stabilized vaccine composition comprises a liquid solution and an alum suspension.

Embodiment I-27. The stabilized vaccine composition of embodiment I-26, wherein the alum suspension comprises or consists of particles with a D50 value of between about 1 μm and about 10 μm.

Embodiment I-28. A method of inducing a protective immune response against an infectious pathogen in a subject, comprising administering the stabilized vaccine composition of any one of embodiments I-1 to I-27 to the subject.

Embodiment I-29. The method of embodiment I-28, wherein the infectious pathogen is of S. pneumoniae or S. pyogenes.

Embodiment I-30. A method for immunizing a subject against pneumococcal infection, comprising administering the stabilized vaccine composition of any one of embodiments I-7 to I-10 or I-12 to I-27 to the subject.

Embodiment I-31. Use of the stabilized vaccine composition of any one of embodiments I-1 to I-27 in the manufacture of a medicament for inducing a protective immune response against S. pneumoniae or S. pyogenes.

Embodiment I-32. Use of the stabilized vaccine composition of any one of embodiments I-7 to I-10 or I-12 to I-27 in the manufacture of a medicament for inducing a protective immune response against pneumococcal infection in a subject.

Embodiment I-33. A stabilized vaccine composition of any one of embodiments I-1 to I-27 for use in inducing a protective immune response against an infectious pathogen in a subject.

Embodiment I-34. The stabilized vaccine composition for use of embodiment I-33, wherein the infectious pathogen is S. pneumoniae or S. pyogenes.

Embodiment I-35. A method of stabilizing a vaccine composition comprising mixing a non-aluminum phosphate salt with a solution comprising:
a. at least one polypeptide-polysaccharide conjugate, wherein the polypeptide component of the at least one polypeptide-polysaccharide conjugate comprises at least two non-natural amino acids (nnAAs);
b. an aluminum adjuvant; and
c. sodium chloride;
wherein the concentration of sodium chloride is less than 200 mM, and the pH is between about 5.5 and 7.

The foregoing description, for purposes of explanation, used specific nomenclature to provide a thorough understanding of the invention. However, it will be apparent to one skilled in the art that specific details are not required in order to practice the invention. Thus, the foregoing descriptions of specific embodiments of the invention are presented for purposes of illustration and description. They are not intended to be exhaustive or to limit the invention to the precise forms disclosed; obviously, many modifications and variations are possible in view of the above teachings. The embodiments were chosen and described in order to explain the principles of the invention and its practical applications, they thereby enable others skilled in the art to utilize the invention and various embodiments with various modifications as are suited to the particular use contemplated. It is intended that the following claims and their equivalents define the scope of the invention

### EXAMPLES

### Example 1: Formulation of Stabilized Vaccine Compositions

A test vaccine formulation (Vax-2) was produced comprising multiple distinct polypeptide-polysaccharide conjugates. The polypeptide component was a polypeptide of SEQ ID NO: 2. VAX-2 contained the polypeptide-polysaccharide conjugates of serotypes 1, 2, 3, 4, 5, 6A, 6B, 7F, 8, 9N, 9V, 10A, 11A, 12F, 14, 15B, 17F, 18C, 19A, 19F, 20B, 22F, 23F, 33F. Diluent buffer, containing succinate, NaCl, sodium phosphate, and PS80 were added to a stainless steel mixing tank. Appropriate amounts of each conjugate were added to the tank by weight and gently mixed using an overhead mixer at a speed of 125±10 rpm for 30±10 min. The mixture was made up to the target volume and the pH was confirmed to be about pH 5.8. This mixture was filtered through three 0.22 μm sterilizing grade filters with PES membranes into a final mixing tank. ALPO was added aseptically and this was further mixed and then filled into 2R glass vials as the final stabilized vaccine composition. The final vaccine composition was stored at 2-8° C.

Manufacture of the stabilized vaccine on larger and smaller scales follows the same procedure as described above with appropriate adjustments in amounts of conjugate, amount of ALPO, size of mixing tank, mixing time, mixing speed, filter size and method of filling glass vials.

### Example 2: Analysis of Stabilized Vaccine Compositions

Stabilized (with sodium phosphate) and non-stabilized (without sodium phosphate) drug products (DP) were manufactured at 2.2 ug/mL concentration. Table 1 shows the composition of these formulations which were manufactured as described in Example 1. They were analyzed for visual appearance, settling properties, and particle size.

TABLE 1
Drug product compositions with and without phosphate salt

| | DS | | | | Sodium | Polysorbate |
|---|---|---|---|---|---|---|
| | conc. | ALPO | Phosphate | Succinate | chloride | 80 (% |
| | (μg/mL) | (mg/mL) | (mM) | (mM) | (mM) | v/v) |
| With | 2.2 | 0.25 | 17 | 5 | 150 | 0.1 |
| phosphate | | | | | | |
| Without | 2.2 | 0.25 | 0 | 5 | 150 | 0.1 |
| phosphate | | | | | | |

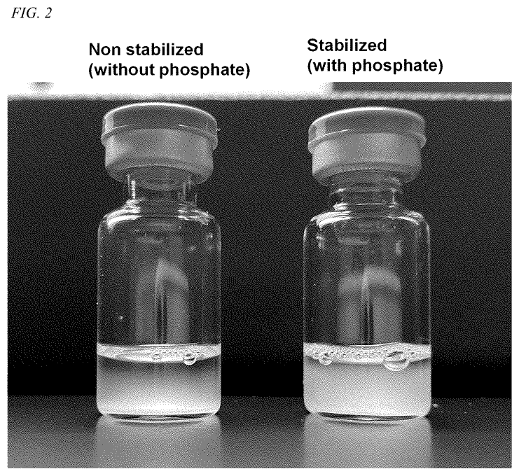

The compositions of Table 1 (drug product with and without phosphate salt) were visually inspected for settling rate. Each vial was shaken for 10-15 seconds and then observed against a light and dark background. FIG. 2 shows the visual effect of phosphate on settling time of the two compositions. Specifically, the DP with sodium phosphate remains suspended after left undisturbed for 60 min while the DP without sodium phosphate showed faster settling.

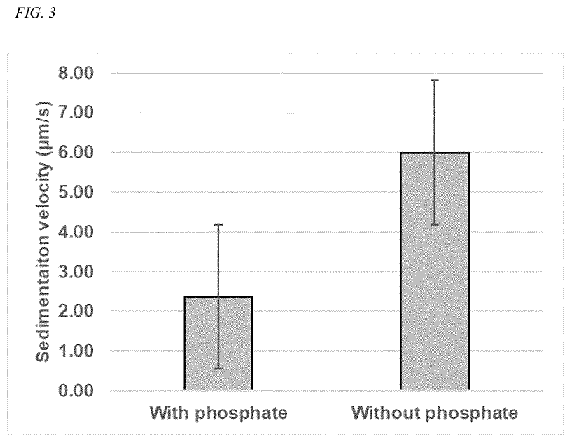

Sedimentation Rate: The settling properties were also analyzed quantitatively using Dispersion Analyzer Lumisizer 650 (LUM). DP with and without sodium phosphate were tested. Vials were shaken for 10-15 seconds prior to testing. 400 μL was pipetted into the Lumisizer cell. The measurement was carried out at 25° C. and centrifugation speed of 200 rpm. Each sample wa9s tested in triplicate. FIG. 3 shows the sedimentation velocity of DP with and without phosphate. Samples with phosphate showed lower values of sedimentation velocity indicating slower settling. On the other hand, samples without phosphate show a high value of sedimentation velocity indicating faster settling. Higher sedimentation rate (faster settling) is an indication of an unstable suspension prone to flocculation. A DP that acts like this can be problematic for homogenous filling of large scale batches and successful clinical dosing protocols.

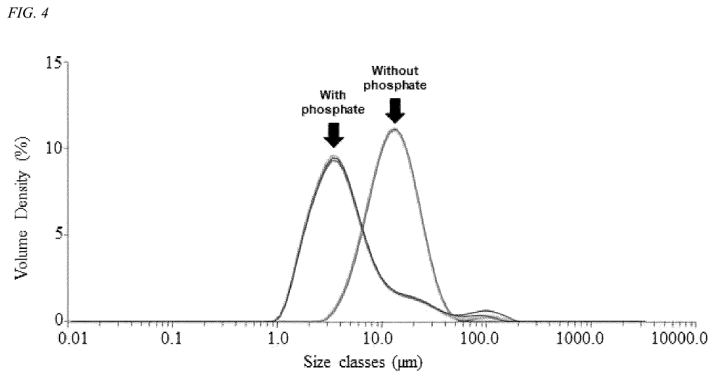

Particle Size: Particle size of the stabilized and non-stabilized DP was measured using Mastersizer 3000 Optical Bench (Malvern). Vials of DP were shaken for 10-15 seconds prior to testing. 0.9% sodium chloride was used as dispersant and added to the Mastersizer cuvette. The obscuration limit was set between 4-8% and stirring speed at 1500 rpm. 200 μl of each sample was added to the cuvette containing the dispersant and mixed. Each measurement was performed 5 times and each sample was tested in triplicates. FIG. 4 shows the particle size distribution of DP with and without phosphate. The presence of phosphate resulted in reduction of particle size of the vaccine. Table 2 also shows that absence of phosphate results in overall larger particles as measured by the D×10, D×50 and D×90 values.

TABLE 2
Drug product compositions with and without phosphate salt

| Sample name | D × 10 (μm) | D × 50 (μm) | D × 90 (μm) |
|---|---|---|---|
| With sodium phosphate | 1.88 | 3.98 | 12.5 |
| Without phosphate | 6.44 | 12.8 | 24.7 |

To further confirm the effect of phosphate on particle size, vials of DP were allowed to settle overnight. These vials were gently mixed by inversion instead of shaking. Measurement conditions were the same as described above. Since these vials were gently inverted instead of vigorous shaking, the mechanical breakdown of the floccules was avoided. A distinct increase in particle size was seen for DP without phosphate at both concentrations for D×10, D×50 and D×90 values. The bigger particle size of the DP without phosphate may indicated flocculation. These data are shown in Table 3.

TABLE 3
Drug product compositions with and without phosphate salt

| Sample name | D × 10 (μm) | D × 50 (μm) | D × 90 (μm) |
|---|---|---|---|
| With sodium phosphate | 2.184 | 5.084 | 23.38 |
| Without phosphate | 27.7 | 64.12 | 143.6 |

### Example 3: Long-Term Stability of Stabilized Vaccine Compositions

Tables 4 and 5 show long-term stability data of a full scale DP batch with 17 mM phosphate. The data was collected after storage at both 2-8° C. (Table 5) and 25° C. (Table 6) using the Mastersizer 3000 Optical Bench (Malvern). The measurement procedure was as described in Example 2. The particle size is generally constant for both temperatures, at all timepoints, indicating the stabilizing effect of phosphate on the DP. The visual appearance of all the test points for both temperatures was homogenous.

TABLE 4
Stabilized vaccine compositions - stability data at 2-8° C.

| | Time point [month] | | | | |
|---|---|---|---|---|---|
| Component | 0 | 1 | 2 | 3 | 6 |
| Particle size D × (10), sample | 2.48 | 2.17 | NT | 2.04 | 2.26 |
| preparation 1 | | | | | |
| Particle size D × (10), sample | 2.48 | 2.10 | | 2.40 | 2.23 |
| preparation 2 | | | | | |
| Particle size D × (10), sample | 2.39 | 1.84 | | 2.33 | 2.25 |
| preparation 3 | | | | | |
| Mean particle size, D × (10) | 2.45 | 2.04 | | 2.25 | 2.25 |
| Particle size D × (50), sample | 4.54 | 3.88 | | 3.64 | 4.10 |
| preparation 1 | | | | | |
| Particle size D × (50), sample | 4.47 | 3.75 | | 4.29 | 3.98 |
| preparation 2 | | | | | |
| Particle size D × (50), sample | 4.42 | 3.16 | | 4.08 | 4.07 |
| preparation 3 | | | | | |
| Mean particle size, D × (50) | 4.48 | 3.60 | | 4.00 | 4.05 |
| Particle size D × (90), sample | 8.57 | 7.18 | | 6.94 | 8.02 |
| preparation 1 | | | | | |
| Particle size D × (90), sample | 8.19 | 6.95 | | 8.42 | 7.04 |
| preparation 2 | | | | | |
| Particle size D × (90), sample | 8.30 | 5.24 | | 7.01 | 7.60 |
| preparation 3 | | | | | |
| Mean particle size, D × (90) | 8.35 | 6.46 | | 7.46 | 7.55 |

TABLE 5
Stabilized vaccine compositions - stability data at 25° C.

| | Time point [month] | | | | |
|---|---|---|---|---|---|
| Component | 0 | 1 | 2 | 3 | 6 |
| Particle size D × (10), sample | 2.48 | 2.10 | 2.19 | 2.26 | 2.06 |
| preparation 1 | | | | | |
| Particle size D × (10), sample | 2.48 | 2.12 | 2.17 | 2 28 | 2.18 |
| preparation 2 | | | | | |
| Particle size D × (10), sample | 2.39 | 2.07 | 2.12 | 2.20 | 2.13 |
| preparation 3 | | | | | |
| Mean particle size, D × (10) | 2.45 | 2.09 | 2.16 | 2.25 | 2.12 |
| Particle size D × (50), sample | 4.54 | 3.70 | 3.91 | 4.08 | 3.69 |
| preparation 1 | | | | | |
| Particle size D × (50), sample | 4.47 | 3.78 | 3.83 | 4.15 | 3.96 |
| preparation 2 | | | | | |
| Particle size 0 × (50), sample | 4.42 | 3.66 | 3.77 | 3.94 | 3.78 |
| preparation 3 | | | | | |
| Mean particle size, D × (50) | 4.48 | 3.72 | 3.84 | 4.05 | 3.81 |
| Particle size D × (90), sample | 8.57 | 6.38 | 7.92 | 7.72 | 6.52 |
| preparation 1 | | | | | |
| Particle size 0 × (90), sample | 8.19 | 7.15 | 6.86 | 8.10 | 8.50 |
| preparation 2 | | | | | |
| Particle size D × (90), sample | 8.30 | 6.47 | 6.81 | 6.97 | 6.59 |
| preparation 3 | | | | | |
| Mean particle size, D × (90) | 8.35 | 6.67 | 7.20 | 7.60 | 7.20 |

### Example 4: Binding of Protein to ALPO in the Presence and Absence of Phosphate

In order to test the effect of phosphate on protein binding to ALPO, small-scale manufacture of stabilized and non-stabilized vaccine compositions was performed as follows. The diluent buffers, containing 5 mM succinate, 150 mM NaCl, 0.100 PS80 with or without 16 mM potassium phosphate were manufactured and added to glass beakers. Appropriate amounts of each DS were added to the containers to get a final conc. of 2.2 μg/mL. The mixtures were gently mixed with magnetic stir bar. They were made up to target volume and the pH was adjusted to be ˜pH 5.8. Then ALPO was added and the resulting suspensions were mixed. These were filled into glass vials as the final DP. This DP was tested using Modified Lowry assay to test for the percent of protein bound to ALPO. Table 6 shows the effect of phosphate on percent binding of the protein to ALPO.

TABLE 6
Effect of phosphate on protein binding to ALPO

| Sample | % Bound protein | % Unbound protein |
|---|---|---|
| With phosphate | 37.4 | 62.6 |
| Without phosphate | 66.1 | 33.9 |

### Example 5: Production of Stabilized Vaccine Formulations

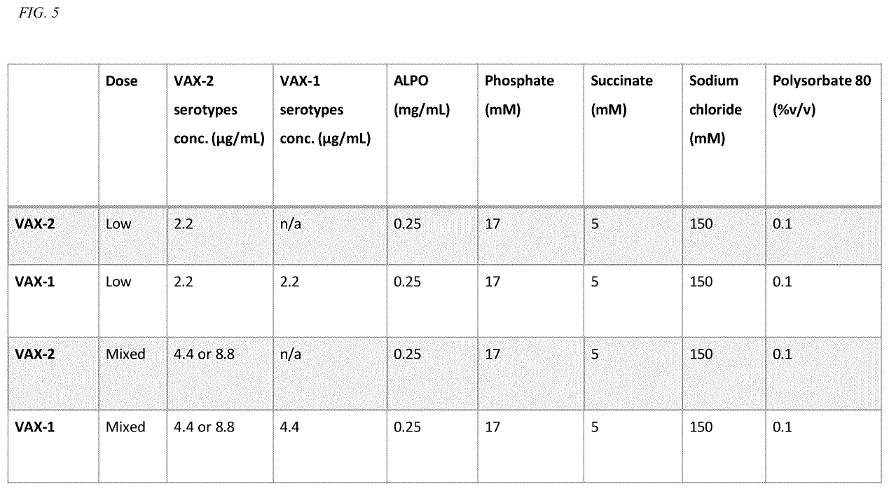

Two test vaccine formulations (Vax-1 and Vax-2) were produced comprising multiple distinct polypeptide-polysaccharide conjugates. The polypeptide component was a polypeptide of SEQ ID NO: 2. VAX-2 contained the following polypeptide-polysaccharide conjugates of serotypes 1, 2, 3, 4, 5, 6A, 6B, 7F, 8, 9N, 9V, 10A, 11A, 12F, 14, 15B, 17F, 18C, 19A, 19F, 20B, 22F, 23F, 33F. VAX-1 contained distinct polypeptide-polysaccharide conjugates of the 24 serotypes above plus seven additional serotypes: 7C, 15A, 16F, 23A, 23B, 31 and 35B. The detailed composition of stabilized vaccines for VAX-1 and VAX-2 are shown in FIG. 5. These vaccines were manufactured as follows: Diluent buffer, containing succinate, NaCl, sodium phosphate, and PS80 was manufactured and added to a 50 ml conical tube. Appropriate amounts of each DS were added to it by volume and gently mixed by inversion. The mixture was made up to the target volume and the pH was confirmed to be about pH 5.8±0.1. ALPO was added to this mixture and this was further mixed and then filled into 2R glass vials as the final stabilized vaccine composition. The final vaccine composition was stored at 2-8° C.

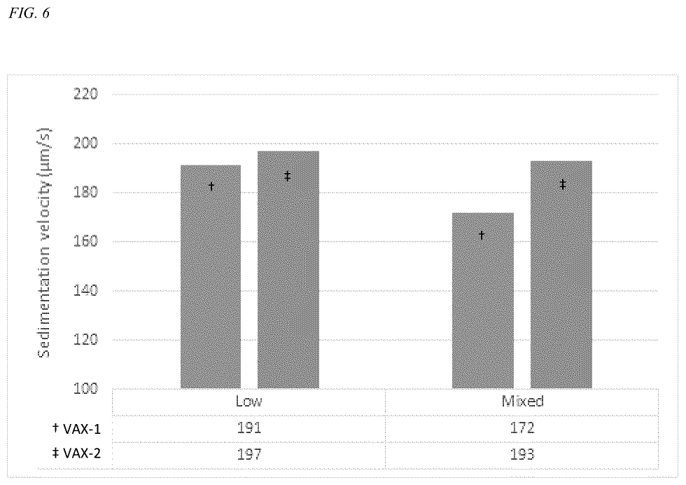

The settling properties of the two test vaccine formulations were then determined using Dispersion Analyzer Lumisizer 650 (LUM). Vials were shaken for 10-15 seconds prior to testing. 270 μL was pipetted into the Lumisizer cell. The measurement was carried out at 25° C. and centrifugation speed of 2500 rpm. FIG. 6 shows the sedimentation velocity of Vax-1 and Vax-2 at Low and Mixed dose. At both low and mixed doses, the sedimentation behavior of VAX-1 is comparable to that of VAX-2.

TABLE 7
Exemplary Polypeptide Sequences

| Seq ID | Name/description | Amino acid sequence |
|---|---|---|
| 1 | CRM197 | MGADDVVDSSKSFVMENFSSYHGTKPGYVDSIQKGIQKPKSGTQ |
| | | GNYDDDWKEFYSTDNKYDAAGYSVDNENPLSGKAGGVVKVTYPG |
| | | LTKVLALKVDNAETIKKELGLSLTEPLMEQVGTEEFIKRFGDGA |
| | | SRVVLSLPFAEGSSSVEYINNWEQAKALSVELEINFETRGKRGQ |
| | | DAMYEYMAQACAGNRVRRSVGSSLSCINLDWDVIRDKTKTKIES |
| | | LKEHGPIKNKMSESPNKTVSEEKAKQYLEEFHQTALEHPELSEL |
| | | KTVTGTNPVFAGANYAAWAVNVAQVIDSETADNLEKTTAALSIL |
| | | PGIGSVMGIADGAVHHNTEEIVAQSIALSSLMVAQAIPLVGELV |
| | | DIGFAAYNFVESIINLFQVVHNSYNRPAYSPGHKTQPFLHDGYA |
| | | VSWNTVEDSIIRTGFQGESGHDIKITAENTPLPIAGVLLPTIPG |
| | | KLDVNKSKTHISVNGRKIRMRCRAIDGDVTFCRPKSPVYVGNGV |
| | | HANLHVAFHRSSSEKIHSNEISSDSIGVLGYQKTVDHTKVNSKL |
| | | SLFFEIKS |
| 2 | Modified CRM- | MGADDVVDSSKSFVMENFSSYHGTKPGYVDSIQXGIQKPKSGTQ |
| | pAMF6 | GNYDDDWKEFYSTDNKYDAAGYSVDNENPLSGKAGGVVKVTYPG |
| | (X = pAMF) | LTKVLALKVDNAETIKKELGLSLTEPLMEQVGTEEFIKRFGDGA |
| | | SRVVLSLPFAEGSSSVEYINNWEQAKALSVELEINFETRGKRGQ |
| | | DAMYEYMAQACAGNRVRNSVGSSLSCINLDWDVIRDXTKTKIES |
| | | LKEHGPIKNKMSESPNKTVSEEKAXQYLEEFHQTALEHPELSEL |
| | | XTVTGTNPVFAGANYAAWAVNVAQVIDSETADNLEKTTAALSIL |
| | | PGIGSVMGIADGAVHHNTEEIVAQSIALSSLMVAQAIPLVGELV |
| | | DIGFAAYNFVESIINLFQVVHNSYNRPAYSPGHXTQPFLHDGYA |
| | | VSWNTVEDSIIRTGFQGESGHDIKITAENTPLPIAGVLLPTIPG |
| | | KLDVNKSKTHISVNGRKIRMRCRAIDGDVTFCRPKSPVYVGNGV |
| | | HANLHVAFHRSSSEKIHSNEISSDSIGVLGYQKTVDHTKVNSXL |
| | | SLFFEIKS |

1. A stabilized vaccine composition comprising:
a. at least one polypeptide-polysaccharide conjugate, wherein the polypeptide component of the at least one polypeptide-polysaccharide conjugate comprises at least two non-natural amino acids (nnAA);
b. an aluminum adjuvant;
c. a non-aluminum phosphate salt; and
d. sodium chloride;
wherein the concentration of sodium chloride is less than 200 mM, and the pH is between about 5.5 and 7.

2. The stabilized vaccine composition of claim 1, further comprising an emulsifier.

3. The stabilized vaccine composition of claim 1, wherein the polypeptide component of the at least one polypeptide-polysaccharide conjugate is a substituted polypeptide antigen.

4. The stabilized vaccine composition of claim 1, wherein the polypeptide component of the at least one polypeptide-polysaccharide conjugate is a carrier protein.

5. The stabilized vaccine composition of claim 1, wherein the polysaccharide component of the at least one polypeptide-polysaccharide conjugate is a capsular polysaccharide.

6. The stabilized vaccine composition of claim 5, wherein the capsular polysaccharide is a capsular polysaccharide of Streptococcus pneumoniae, Neisseria meningitidis, Haemophilus influenzae, or Streptococcus pyogenes.

7. The stabilized vaccine composition of claim 5, wherein the capsular polysaccharide is a capsular polysaccharide of Streptococcus pneumoniae.

8. The stabilized vaccine composition of claim 1, wherein the at least one polypeptide-polysaccharide conjugate comprises at least 20 distinct polypeptide-polysaccharide conjugates.

9. The stabilized vaccine composition of claim 8, wherein the at least 20 distinct polypeptide-polysaccharide conjugates comprise at least 24 distinct polypeptide-polysaccharide conjugates; wherein there is a distinct polypeptide-polysaccharide conjugate comprising a capsular polysaccharide for each of Streptococcus pneumoniae serotypes 1, 2, 3, 4, 5, 6A, 6B, 7F, 8, 9V, 9N, 10A, 11A, 12F, 14, 15B, 17F, 18C, 19A, 19F, 20B, 22F, 23F, and 33F.

10. The stabilized vaccine composition of claim 8, wherein the at least 20 distinct polypeptide-polysaccharide conjugates comprise at least 31 distinct polypeptide-polysaccharide conjugates; wherein there is a distinct polypeptide-polysaccharide conjugate comprising a capsular polysaccharide for each of Streptococcus pneumoniae serotypes 1, 2, 3, 4, 5, 6A, 6B, 7C, 7F, 8, 9V, 9N, 10A, 11A, 12F, 14, 15A, 15B, 16F, 17F, 18C, 19A, 19F, 20B, 22F, 23A, 23B, 23F, 31, 33F, and 35B.

11. The stabilized vaccine composition of claim 1, wherein the polysaccharide component of the at least one polypeptide-polysaccharide conjugate is a cell wall polysaccharide.

12. The stabilized vaccine composition of claim 1, further comprising at least one unsubstituted polypeptide antigen.

13. The stabilized vaccine composition of claim 1, wherein the at least two nnAAs are selected from the group consisting of 2-amino-3-(4-azidophenyl)propanoic acid (pAF), 2-amino-4-azidobutanoic acid, 2-azido-3-phenylpropionic acid, 2-amino-3-azidopropanoic acid, 2-amino-3-(4-(azidomethyl)phenyl)propanoic acid (pAMF), 2-amino-3-(5-(azidomethyl)pyridin-2-yl)propanoic acid, 2-amino-3-(4-(azidomethyl)pyridin-2-yl)propanoic acid, 2-amino-3-(6-(azidomethyl)pyridin-3-yl)propanoic acid, and 2-amino-5-azidopentanoic acid.

14. The stabilized vaccine composition of claim 1, wherein the at least two nnAAs are pAMF.

15. The stabilized vaccine composition of claim 1, wherein the polypeptide component of the polypeptide-polysaccharide conjugate comprises 3-8 nnAA and the nnAA present are all the same nnAA.

16. The stabilized vaccine composition of claim 1 wherein the polypeptide component of the polypeptide-polysaccharide conjugate comprises an amino acid sequence that is at least 80% identical to SEQ ID NO: 1.

17. The stabilized vaccine composition of claim 1, wherein the polypeptide component of the polypeptide-polysaccharide conjugate comprises an amino acid sequence that is at least 90% or 95% identical to SEQ ID NO: 1.

18. The stabilized vaccine composition of claim 1, wherein the polypeptide component of the polypeptide-polysaccharide conjugate comprises or consists of the amino acid sequence of SEQ ID NO: 2.

19. The stabilized vaccine composition of claim 1, wherein the aluminum adjuvant is aluminum phosphate (ALPO), aluminum hydroxide (ALOH), amorphous aluminum hydroxyphosphate sulfate, or aluminum potassium phosphate.

20. The stabilized vaccine composition of claim 1, wherein the aluminum adjuvant is ALPO.

21. The stabilized vaccine composition of claim 1, wherein the concentration of the aluminum adjuvant is between about 125 μg/mL and about 250 μg/mL.

22. The stabilized vaccine composition of claim 1, wherein the concentration of the non-aluminum phosphate salt is between about 15 mM and 20 mM.

23. The stabilized vaccine composition of claim 1, wherein the concentration of the non-aluminum phosphate salt is about 17 mM.

24. The stabilized vaccine composition of claim 1, wherein the concentration of sodium chloride is about 150 mM.

25. The stabilized vaccine composition of claim 1, wherein the pH is about 5.8.

26. The stabilized vaccine composition of claim 1, wherein the stabilized vaccine composition comprises a liquid solution and an alum suspension.

27. The stabilized vaccine composition of claim 26, wherein the alum suspension comprises or consists of particles with a D50 value of between about 1 μm and about 10 μm.

28. A method of inducing a protective immune response against an infectious pathogen in a subject, comprising administering the stabilized vaccine composition of claim 1 to the subject.

29. The method of claim 28, wherein the infectious pathogen is of S. pneumoniae or S. pyogenes.

30. A method for immunizing a subject against pneumococcal infection, comprising administering the stabilized vaccine composition of claim 7 to the subject.

31.-34. (canceled)

35. A method of stabilizing a vaccine composition comprising mixing a non-aluminum phosphate salt with a solution comprising:
a. at least one polypeptide-polysaccharide conjugate, wherein the polypeptide component of the at least one polypeptide-polysaccharide conjugate comprises at least two non-natural amino acids (nnAAs);
b. an aluminum adjuvant; and
c. sodium chloride;
wherein the concentration of sodium chloride is less than 200 mM, and the pH is between about 5.5 and 7.


SEQUENCE LISTING (2 sequences)

SEQ ID NO: 1 - protein, 536 aa, synthetic construct
MGADDVVDSSKSFVMENFSSYHGTKPGYVDSIQKGIQKPKSGTQGNYDDDWKEFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFLHDGYAVSWNTVEDSIIRTGFQGESGHDIKITAENTPLPIAGVLLPTIPGKLDVNKSKTHISVNGRKIRMRCRAIDGDVTFCRPKSPVYVGNGVHANLHVAFHRSSSEKIHSNEISSDSIGVLGYQKTVDHTKVNSKLSLFFEIKS

SEQ ID NO: 2 - protein, 536 aa, synthetic construct
  SITE 34: note = Xaa is 2-amino-3-(4-(azidomethyl)phenyl)propanoic acid
  SITE 213: note = Xaa is 2-amino-3-(4-(azidomethyl)phenyl)propanoic acid
  SITE 245: note = Xaa is 2-amino-3-(4-(azidomethyl)phenyl)propanoic acid
  SITE 265: note = Xaa is 2-amino-3-(4-(azidomethyl)phenyl)propanoic acid
  SITE 386: note = Xaa is 2-amino-3-(4-(azidomethyl)phenyl)propanoic acid
  SITE 527: note = Xaa is 2-amino-3-(4-(azidomethyl)phenyl)propanoic acid
MGADDVVDSSKSFVMENFSSYHGTKPGYVDSIQXGIQKPKSGTQGNYDDDWKEFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRNSVGSSLSCINLDWDVIRDXTKTKIESLKEHGPIKNKMSESPNKTVSEEKAXQYLEEFHQTALEHPELSELXTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHXTQPFLHDGYAVSWNTVEDSIIRTGFQGESGHDIKITAENTPLPIAGVLLPTIPGKLDVNKSKTHISVNGRKIRMRCRAIDGDVTFCRPKSPVYVGNGVHANLHVAFHRSSSEKIHSNEISSDSIGVLGYQKTVDHTKVNSXLSLFFEIKS